(19) United States
(12) Patent Application Publication
(10) Pub. No.: US 2019/0381236 A1
(43) Pub. Date: Dec. 19, 2019

(54) AUTOINJECTOR CARRIER

(71) Applicant: Omar-Samir M. Badr, Pooler, GA (US)

(72) Inventor: Omar-Samir M. Badr, Pooler, GA (US)

(21) Appl. No.: 16/011,431

(22) Filed: Jun. 18, 2018

(51) Int. Cl. A61M 5/00; A61J 1/03

(52) U.S. Cl. CPC A61M 5/002; A61J 1/035; A61J 2205/50; A61M 2209/088; A61M 2205/583

(57) ABSTRACT

An emergency device carrier includes a separate compartment for holding a supplemental treatment article. As one example of many, a primary compartment may be configured to receive an autoinjector, and the supplemental treatment article may be a packaged antihistamine such as a blister-packaged antihistamine. The separate compartment may comprise a slot or sleeve on the front surface of the primary compartment, and a tile configured to be received by the slot or sleeve. The tile may include a back surface with a cavity to receive the supplemental treatment article; that is, the cavity may be shaped to receive a round or oval-shaped antihistamine tablet. The slot or sleeve may comprise one or more side and bottom tabs, such that when the tile is received in the slot or sleeve, the front surface of the tile is exposed. The carrier may additionally include a structure that facilitates coupling to a belt.

### FIELD OF THE INVENTION

This invention relates generally to medical device carriers and, in particular, to a carrier that may be used to hold an epinephrine autoinjector with a separate compartment for carrying a supplemental treatment drug, article or device.

### BACKGROUND OF THE INVENTION

Anaphylaxis is a rapid, serious allergic reaction that can be life-threatening. Symptoms may include an itchy rash, throat or tongue swelling, shortness of breath, vomiting, lightheadedness, or low blood pressure. Causes may include insect bites or stings, as well as certain foods or medications. The reaction involves the release of mediators from certain types of white blood cells triggered by either immunologic or non-immunologic mechanisms.

A primary treatment of anaphylaxis is epinephrine (adrenaline) injection into a muscle. Carrying an epinephrine autoinjector and identification regarding the condition is recommended in people with a history of such allergic reactions.

An epinephrine autoinjector delivers a measured dose or doses of epinephrine. There are now several auto-injectors on the market, including Auvi-Q (Kaléo), EpiPen (Mylan); Impax Therapeutics (generic version of the branded Adrenaclick device); and Mylan USP auto-injector (generic version of the branded EpiPen). One of the devices—the Auvi-Q—includes voice-guided instructions.

Following a diagnosis confirming the presence or likely presence of anaphylaxis, the common protocol for the treatment of anaphylaxis includes placing the patient in a recumbent position with the lower extremities elevated while monitoring vital signs. Epinephrine is then administered, with dosage being selected on the basis of patient weight. Oxygen may be administered, if available, and the airway may be maintained with an oropharyngeal device.

The protocol for anaphylaxis treatment also includes administering an antihistamine (i.e., H1 blocker) to relieve mild allergy symptoms such as itching, sneezing, hives and rashes. Medications in this class include diphendydramine (Benadryl®) and cetirizine (Zyrtec®). To be sure, antihistamines should not be used instead of epinephrine as they cannot stop or control a severe reaction. However, once a severe reaction is under control, a drug such as Benadryl can help manage certain symptoms.

While there are carriers for epinephrine autoinjectors, they tend to be soft pouches without any special compartments for additional supplies such as antihistamine tablets. Thus, the need remains for an epinephrine autoinjector carrier that address more the needs of a person suffering from an anaphylaxis.

### SUMMARY OF THE INVENTION

This invention improves upon the existing art by providing an emergency device carrier with a separate compartment for holding a supplemental treatment article. In preferred embodiments, the emergency device carrier is adapted to carry an emergency medical device such as an autoinjector. In such an embodiment, the carrier includes a primary compartment configured to receive the autoinjector, with a top opening corresponding to the cross section of the autoinjector, such that the autoinjector is received within the primary compartment with a snug, frictional fit, and with the top of the autoinjector protruding from the top opening thereof.

The primary compartment has a front surface and a bottom surface, and a portion of the front surface and the bottom surface may be cut out, enabling a user to push up on the bottom surface of the autoinjector when received within the primary compartment to release the autoinjector from the top opening in the primary compartment.

The front surface of the primary compartment preferably further includes a secondary compartment for holding a supplemental treatment article related to the purpose of the autoinjector. As one example, the autoinjector may be an epinephrine autoinjector, and the supplemental treatment article may be a packaged antihistamine such as a blister-packaged antihistamine.

The secondary compartment may further comprises a slot or sleeve on the front surface of the primary compartment, and a tile configured to be received by the slot or sleeve. The tile has a front surface, and may include a back surface with a cavity to receive the supplemental treatment article. The cavity may be shaped to receive a round or oval-shaped antihistamine tablet.

The slot or sleeve may comprise one or more side and bottom tabs, such that when the tile is received in the slot or sleeve, the front surface of the tile is exposed. The exposed, front surface of the tile includes textual or graphical indicia. The primary compartment may additionally include a back surface with a structure that facilitates coupling to a belt.

### BRIEF DESCRIPTION OF THE DRAWINGS

FIG. 1 is an oblique, exploded view of a preferred embodiment of the invention;

FIG. 2 is a front view of the embodiment of FIG. 1; and

FIG. 3 is a back view of the embodiment of FIG. 1.

### DETAILED DESCRIPTION OF THE PREFERRED EMBODIMENTS

This invention improves upon the existing art of epinephrine autoinjector carriers by providing a carrier with a separate compartment for carrying a supplemental treatment article. In the preferred embodiment, the carrier includes a primary compartment configured to hold an Auvi-Q epinephrine autoinjector, and a secondary compartment to hold a Benadryl tablet. However, the invention is not limited in this regard, as the carrier may be configured to hold other types of autoinjectors, including cylindrical EpiPen type injectors, as well as other types of drugs for other emergency conditions such as Naloxone injectors, and other supplemental drugs and/or supplies. The shape and dimensions of all such injectors are well know and/or readily available; see, for instance, www.auvi-q.com, the contents of which are incorporated herein by reference.

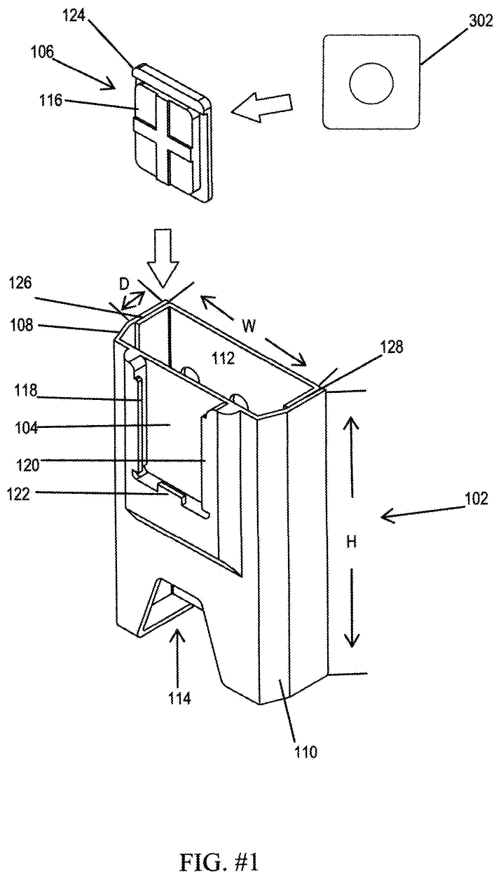

FIG. 1 is an oblique, exploded view of a preferred embodiment of the invention, which includes a primary compartment 102 and a secondary compartment in the form of slot or sleeve 104 configured to receive a supplemental article such as a Benadryl tablet. The primary compartment shown is configured to hold an Auvi-Q autoinjector, which is a generally rectangular unit having a height on the order of 3⅜″, a width on the order of 2″, and a thickness of about ⅝″. The current Auvi-Q autoinjector unit also has somewhat rounded front side edges.

Given the dimensions of an existing Auvi-Q epinephrine autoinjector, the primary compartment 102 has a width W of about 2″ and a depth D of about ⅝″. The height of the primary compartment 102 is less than the height of the Auvi-Q unit, so that the top portion is exposed at the top of the carrier. Accordingly, the height H is in the range of 2.5 to 3 inches. The front, side edges 108, 110 are preferably truncated as shown for better conformity with the rounded front side edges of the Auvi-Q unit.

Generally speaking, the inside dimensions of the primary carrier 102 are such that the autoinjector is received with a snug, frictional fit that facilitates controlled, unimpeded release through the top opening 112 while preventing the autoinjector from inadvertently falling out. In the preferred embodiment, the primary compartment 102 further includes a front bottom cut-out 114, such that a portion of the front bottom of the autoinjector unit is visible while allowing a user to push up on the bottom surface of the autoinjector unit so that it is released from the top opening 112.

Continuing the reference to FIG. 1, the primary compartment 102 preferably includes a front slot or sleeve 104 configured to receive a secondary carrier that may be in the form of a tile 106. The tile 106 has a front surface 116 that may include visual indicia such as the “red cross” symbol shown, or other optional textual or graphical materials.

The tile 106 is dimensioned to fit into the slot or sleeve 104, and is frictionally held in position with side and bottom tabs 118, 120, 122. In the preferred configuration, the tabs do not cover the front of the tile so that the textual/graphical indicia on the front 116 of the tile, if present, are visible when the tile 106 is received by the front slot or sleeve 104. The textual/graphical indicia may have a raised surface or lettering to enhance the traction of a finger used to release the tile.

Note that the top of tile 116 may include a top front outwardly extending pull ridge 124 to facilitate removal of the tile with a fingertip or fingernail. Again, however, as with the primary carrier, the dimensions of the front slot or sleeve 104 and tabs 118, 120, 122 are such that the tile 106 is received with a snug, frictional fit that facilitates controlled, unimpeded release of the tile while preventing the tile from inadvertently falling out.

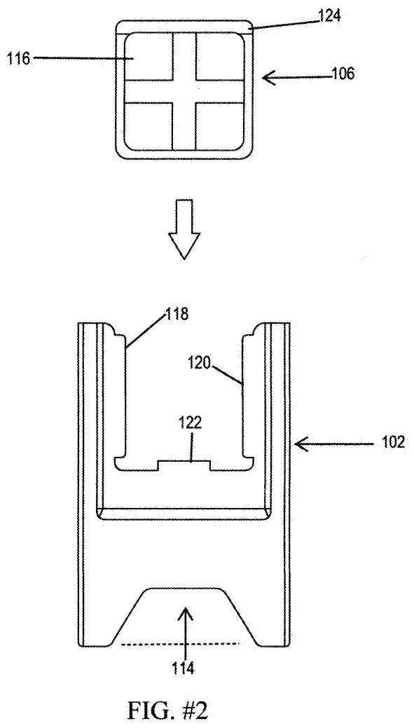

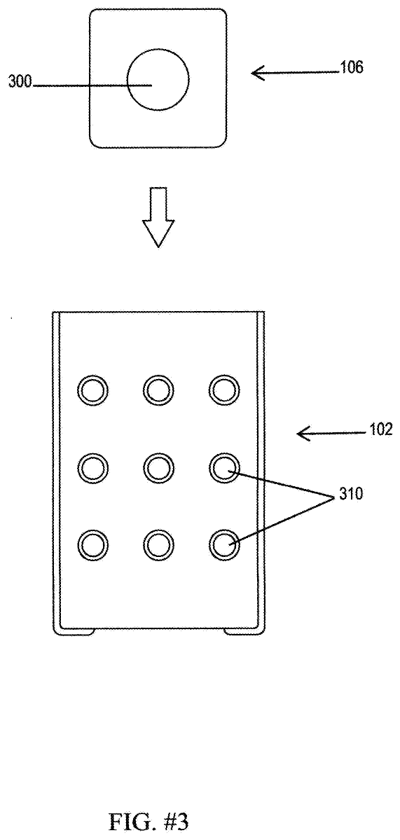

FIG. 2 is a front view of the preferred embodiment, and FIG. 3 is a back view, which may optionally include apertures 310. Such apertures may be used to couple the carrier to various other structures for mounting to a person including belt-mounting, mounting to other equipment including bicycles, etc. Alternatively, the back side of the carrier may have a permanent or integrally formed loops, slots or sleeves for any type of personal, clothing or device attachment.

Also visible in FIG. 3, the secondary carrier or tile 106 includes a cavity 300 to hold a supplemental treatment article such as a Benadryl tablet. For example, in accordance with one embodiment, the cavity may be circular as shown, to receive a Benadryl blister pack 302 (FIG. 1) for children or adults. Both such blister packs have generally square or rectangular outer dimensions on the order of 1 to 1.35 inches, with a central tablet-holding round or oval compartment with a maximum dimension of about 0.5 inches and a thickness on the order of 0.2-0.225 inches.

Given the dimensions of the child/adult Benadryl blister pack, the outer dimensions (height/width) of the tile 106 are preferably the same or slightly larger than the outmost dimensions of the blister pack 302, with the dimensions of the cavity 300 being at least large enough the accommodate the dimensions of the child/adult tablet itself.

In the preferred embodiments, the entire carrier, including the primary compartment 102 and tile 106, are constructed from plastic, and may be injected molded, thermally formed, or the like. The various dimensions and wall thicknesses are such that the carrier is at least semi-rigid with some flexibility to allow for autoinjector insertion and removal, while the tile 106 may be at least semi-rigid if not rigid in construction.

1. A carrier for carrying an emergency medical device such as an autoinjector, and wherein the autoinjector has a top and a bottom defining a height, and a width and depth defining a cross section, the carrier comprising:
a primary compartment configured to receive the autoinjector, the primary compartment having a height less than that of the autoinjector, and a top opening corresponding to the cross section of the autoinjector, such that the autoinjector is received within the primary compartment with a snug, frictional fit, and with the top of the autoinjector protruding from the top opening thereof;
the primary compartment further having a front surface and a bottom surface, and wherein a portion of the front surface and the bottom surface are cut out, enabling a user to push up on the bottom surface of the autoinjector when received within the primary compartment to release the autoinjector from the top opening in the primary compartment; and
wherein the front surface of the primary compartment further includes a secondary compartment for holding a supplemental treatment article related to the purpose of the autoinjector.

2. The carrier of claim 1, wherein:
the autoinjector is an epinephrine autoinjector; and
the supplemental treatment article is a packaged antihistamine.

3. The carrier of claim 1, wherein:
the autoinjector is a generally rectangular epinephrine autoinjector; and
the supplemental treatment article is a blister-packaged antihistamine.

4. The carrier of claim 1, wherein the secondary compartment further comprises:
a slot or sleeve on the front surface of the primary compartment; and
a tile configured to be received by the slot or sleeve, the tile having a front surface and a back surface with a cavity to receive the supplemental treatment article.

5. The carrier of claim 4, wherein the cavity is adapted to receive a round or oval-shaped antihistamine tablet.

6. The carrier of claim 4, wherein the slot or sleeve comprises one or more side and bottom tabs, such that when the tile is received in the slot or sleeve, the front surface of the tile is exposed.

7. The carrier of claim 5, wherein the exposed, front surface of the tile includes textual or graphical indicia.

8. The carrier of claim 1, wherein the primary compartment includes a back surface with a structure that facilitates coupling to a belt.
